(19) United States
(12) Patent Application Publication
(10) Pub. No.: US 2018/0014556 A1
(43) Pub. Date: Jan. 18, 2018

(54) ANIMAL FEED SUPPLEMENT COMPOSITION

(71) Applicant: VETCARE OY, Salo (FI)

(72) Inventors: Eila JÄRVENPÄÄ, Lieto (FI); Jarkko MÄKINEN, Jokioinen (FI); Tuomo TUPASELA, Tarvasjoki (FI); Jaakko HIIDENHOVI, Forssa (FI); Shea BEASLEY, Espoo (FI); Kalevi HEINONEN, Hautjärvi (FI)

(73) Assignee: VETCARE OY, Salo (FI)

(21) Appl. No.: 15/541,244

(22) PCT Filed: May 27, 2016
(86) PCT No.: PCT/FI2016/050371

§ 371 Date: Jun. 30, 2017

(30) Foreign Application Priority Data
May 29, 2015 (FI) U20154100

(51) Int. Cl. A23K 10/26; A23K 50/40; A23K 50/10; A23K 20/174; A23K 20/158; A61K 35/57; A23K 20/147

(52) U.S. Cl. CPC A23K 10/26; A61K 35/57; A23K 50/40; A23K 20/147; A23K 20/174; A23K 20/158; A23K 50/10

(57) ABSTRACT

The invention relates to an animal feed supplement composition comprising egg fraction of poultry egg, shell membrane fraction and shell fraction. The invention further relates to a method for preparing the composition, in which egg fraction composed of poultry egg yolk and albumen, shell membrane fraction and shell fraction are combined into a homogenous mixture.

### FIELD OF THE INVENTION

The invention relates to a feed supplement composition comprising fractions of poultry eggs, for animals.

### BACKGROUND OF THE INVENTION

Ready-made foods, such as pellets and nuggets, especially intended for companion animals often contain fibre and carbohydrates that needlessly increase the faecal mass of the animal. Ready-made foods do not always take into consideration the special characteristics of the digestive tract of companion animals, such as dogs or cats. Further, the amounts of proteins, minerals, trace elements and vitamins are often low or their ratio is disadvantageous in terms of the nutrition of the animal. For a short small intestine of an animal, it is advantageous that a nutriment is in an adequately compact and easily digestible form. It is also desirable that fats are the primary energy sources instead of carbohydrates.

Lately, allergies and hypersensitivities in animals have increased considerably, and many animal owners attempt to avoid processed animal foods as possible causes of allergies.

Chicken eggs contain many nutritionally valuable components. Raw chicken eggs are given to companion animals to some extent along with their daily nourishment. The use and handling of raw chicken eggs is, however, generally considered difficult and laborious, shells are typically separated from the yolk and albumen and thrown in the waste bin, wherein also the nutrients contained in the shells and shell membranes remain unused and the shells end up in the refuse pit. Chicken eggs have limited preservability. A raw chicken egg may contain pathogens, such as salmonella, campylobacteria and viruses, which have been shown to transfer from a pet to family members. Additionally, the ratio of nutritionally valuable components in eggs is not ideal for all pets.

The use of chicken eggshell membranes has been researched especially in the treatment of joint problems in mammals. Publication US 2011117207 A discloses a composition intended for the relief of joint pains, which contains eggshell membrane and astaxanthin. US 2013337080 A discloses a composition to prevent cartilage degradation, which composition contains concentrated eggshell membrane.

On the basis of the above, it can be seen that there is a need to provide a feed supplement composition, which is especially suitable for animals, preserves well and is based on chicken egg.

### SUMMARY OF THE INVENTION

The object of the invention is to obviate or substantially decrease the disadvantages presented above, which relate to animal nutriments and the use of fresh chicken eggs as an animal feed supplement, and to find a new chicken egg-based composition that is suitable as a feed supplement composition for animals.

Further, the object of the invention is a safe chicken egg-based feed supplement composition having an adequate preservability.

The invention relates more generally to a feed supplement composition for animals, which comprises poultry egg fractions.

Specifically, the invention relates to a feed supplement composition for animals, which comprises 80-98 wt % of egg fraction of poultry egg, 0.1-15 wt % of shell membrane fraction and 1-10 wt % of shell fraction.

The invention relates also to a method for preparing a feed supplement composition for animals, in which method 80-98 wt % of egg fraction composed of poultry egg yolk and albumen, 0.1-15 wt % of shell membrane fraction and 1-10 wt % of shell fraction are combined into a homogenous mixture.

A feed supplement composition according to the invention can be used for companion animals, fur animals and production animals to support and supplement the basic food of the animal, for allergic animals with the exception of animals allergic to eggs, for young and growing animals, active animals, pregnant animals, nursing animals and aging animals.

The characterizing features of the invention are presented in the accompanying claims.

### FIGURES

FIG. 1 shows the effect of the composition on the mobility and the coat/skin condition of a dog.

FIG. 2 shows the effect of the composition on the desire to play and general mood of a dog.

### DEFINITIONS

Unless otherwise defined, the terms used in the description and in the claims have the meanings, which are generally used in the animal food industry and in animal nutrition fields. The following terms are especially used with the meanings presented below.

The term “poultry” means herein all bird species grown or ranched for the production of meat, eggs or other products, messenger, show and ornamental birds as well as game bird species. By poultry is specifically meant chickens, domestic ducks, geese, turkeys, ostrich birds, such as ostriches and emus, guinea fowl, quails, pigeons, pheasants, partridges, ducks, grouse, capercaillie and peacocks.

### DETAILED DESCRIPTION OF THE INVENTION

It was surprisingly found that a feed supplement composition suitable for animals is obtained from poultry egg fractions by combining in a manner according to the invention, in which composition valuable nutrients are in a ratio suitable for animals, and the composition preserves well, it can be easily and exactly rationed, it is tasty to animals and it is a product containing natural components.

A feed supplement composition according to the invention can be given to animals, which are selected from companion animals, fur animals and production animals. Companion animals are suitably selected from dogs, cats, ferrets, rodents, pigs, turtles, reptiles, such as lizards and snakes, and birds. Fur animals are selected from foxes, minks, chinchillas, nutrias, raccoons, sables, ermines, muskrats and polecats. Production animals are selected from bovines, such as cows and buffalo, hoofed animals, such horses and donkeys, llamas, alpacas, sheep, goats and pigs. The feed supplement composition is especially well suited for supplementing the basic nutriments of dogs and cats.

An animal feed supplement composition according to the invention comprises 80-98 wt % of egg fraction of poultry egg, 0.1-15 wt % of shell membrane fraction and 1-10 wt % of shell fraction. Preferably, the feed supplement composition comprises 91-98 wt % of egg fraction of poultry egg, 0.5-5.5 wt % of shell membrane fraction and 1.5-3.5 wt % of shell fraction. Most preferably, the feed supplement composition comprises 92.5-97 wt % of egg fraction of poultry egg, 1-4 wt % of shell membrane fraction and 2-3.5 wt % of shell fraction.

Preferably, chicken egg fractions are used in the feed supplement composition.

The advantageous Ca/P ratio (the ratio of calcium to phosphorus) of the feed supplement composition is in the range of 1:1-2:1.

Egg Fraction

By egg fraction is meant the fraction composed of yolk and albumen separated from raw eggs. Egg fraction is dried and its water content is typically up to 5 wt %.

The separation of egg fraction from the shell and shell membrane and drying can be performed by using per se known methods. Suitably, drying can be performed by spray drying, wherein the egg fraction is obtained as a fine-grained powder. Egg fraction (dry powder) typically contains 25-40 wt % of albumen and 60-75 wt % of yolk. Egg fraction contains an abundance of unsaturated fatty acids and it is a good source of DHA. Further, egg fraction contains amino acids as well as vitamins A, D, B9 and B5. Yolk serves especially as a phosphorus source. The typical composition of egg fraction separated from chicken egg and dried is shown below in Table 1.

TABLE 1
Typical composition of egg fraction separated from chicken egg

| | Dried egg | | |
|---|---|---|---|
| Wt % of fresh weight | fraction | Yolk | Albumen |
| Proteins | 47.9 | 33.7 | 81.1 |
| Fats | 42.4 | 58.1 | — |
| Carbohydrates* | 2.7 | 2.1 | 7.8 |
| Ash | 3.6 | 3.3 | 5.3 |
| Water | 3.4 | 2.7 | 5.8 |

*calculated

### Shell Membrane Fraction

By shell membrane is meant the membrane between the albumen and the shell of poultry eggs. Shell membrane fraction contains shell membrane, which can be separated from the egg. Shell membrane fraction is dried and it typically contains up to 15 wt % of water. Shell membrane fraction typically contains approximately 35 wt % of collagen, approximately 9 wt % of chondroitin sulfate, approximately 10 wt % of glucosamine and approximately 5-10 wt % of hyaluronic acid relative to its fresh weight. Shell membrane fraction further contains i.a. elastin (desmosine and isodesmosine), dermatan sulfate, keratan sulfate and sialic acid.

The typical composition of shell membrane fraction is shown below in Table 2.

TABLE 2
Typical composition of dried shell membrane fraction

| Compound | wt % |
|---|---|
| Water | 13.9 |
| Protein | 82.1 |
| Fat | 2.7 |
| Carbohydrate | 0.6 |
| Ash (calcium) | 0.6 |

Shell membrane is typically separated from the raw egg along with the shell. Shell membrane can be further separated from the shell using per se known methods, and dried into a shell membrane powder using known methods. The shell and shell membrane can also be treated together, wherein the shell and membrane are dried and ground into a finely-ground powder. The powder can be used as such in the composition.

### Shell Fraction

By shell fraction is herein meant the shell separated from the poultry egg or organic calcium salt or inorganic calcium carbonate and their combinations. The shells separated from the poultry eggs are dried and ground into a finely-ground powder. Shell fraction contains minerals, especially calcium, calcium carbonate (CaCO₃) or calcium salt with an organic acid. Preferably, as shell fraction is used the shells separated from poultry eggs.

The organic calcium salt is suitably calcium salt with an organic acid, preferably calcium lactate or calcium citrate.

The typical composition of shell fraction is shown below in Table 3.

TABLE 3
Typical composition of shell fraction

| Compound | wt % |
|---|---|
| Water | 0.5 |
| Protein | 3.2 |
| Ash | 95.3 |
| Calcium | 37.7 |

An animal feed supplement composition according to the invention comprises egg fraction, shell membrane fraction and shell fraction, which meet the microbiological purity requirements generally set for feeds.

An animal feed supplement composition according to the invention can be rationed as such to an animal or the feed supplement composition can be mixed with one or more additives to prepare the desired product. The additive is preferably an additive in accordance with the laws.

The additive can be selected i.a. from antioxidants, flavouring agents, chondroprotectives, such as glucosamine sulfate, rooster comb extract, shark cartilage extract, chondroitin sulfate, methylsulfonylmethane (MSM) and hyaluronic acid, vitamins and vitamin sources, such as rose hip powder and sea buckthorn powder, the oils and fractions obtained from fish and other seafood, algae and plankton, such as green shell mussel powder, green shell mussel oil, micro algae powder, minerals and trace elements, processing aids, such as enzymes, emulsifiers, binders, carriers, anti-caking agents, tableting adjuvants and fillers, vegetable oils and their combinations.

In one preferred embodiment, into the supplemental feed composition is mixed vitamin C or vitamin E or their combination, which function as both antioxidants and vitamins.

An animal feed supplement composition according to the invention is prepared using a method comprising steps of combining egg fraction composed of 80-98 wt % of poultry egg yolk and albumen, 0.1-15 wt % of shell membrane fraction and 1-10 wt % of shell fraction, wherein a homogeneous mixture is obtained. The mixture is in the form of a homogeneous powder. Preferably, 91-98 wt % of egg fraction of poultry egg, 0.5-5.5 wt % of shell membrane fraction and 1.5-3.5 wt % of shell fraction are used, most preferably 92.5-97 wt % of egg fraction of poultry egg, 1-4 wt % of shell membrane fraction and 2-3.5 wt % of shell fraction.

Optional additives can be mixed into the prepared composition or they can alternatively be mixed into the composition in connection with preparation.

For mixing can be used generally known devices suitable for mixing dry substances.

From the supplemental feed composition can further, if desired, be formed preparations and products suitable for use with animals depending i.a. on the target animal species.

From an animal feed supplement composition according to the invention, into which is optionally mixed additives, can be prepared products comprising the feed supplement composition, which can be in the form of powder, granules, pellets, tablets, capsules, gel, paste, emulsion, liquid or the composition can be mixed into a suitable carrier, wherein from it can be made chew bars, chew bones, chew rolls, (hard or soft) treats, or it can be combined into animal food or drink.

As a carrier can be used generally known carriers used in animal products, of which can be mentioned, for example, gelatins, starches, melibiose, maltodextrins, celluloses, pectins, seaweed hydrocolloids, glycerine, lecithins, oils, fats and natural waxes, such as beeswax. The composition can contain moisture (water). The water may originate from the fractions used in the composition. The moisture content of the composition can vary in the range of 2-10 wt %, typically 3-7 wt %, preferably 4-6 wt %.

An animal feed supplement composition according to the invention is rationed to animals depending on the species of animal, the physiological condition of the animal (this also includes pregnancy and nursing), age, sex, and level of activity. For example, the feed supplement composition can be rationed for a dog according to Table 4, the daily ration for a cat is 2-5 g.

TABLE 4
Typical rationing of feed supplement for a dog

| Weight of dog | | Ration (g) |
|---|---|---|
| <10 | kg | 5-8 |
| 10-45 | kg | 8-16 |
| >45 | kg | 16-30 |

An animal (dog/cat) typically receives 13/10% of its daily protein requirements, 8/6% of its daily energy requirements and on the average 17/30% of its daily vitamin requirements from the feed supplement composition.

### Advantages of the Invention

The present invention has many advantages. In an animal feed supplement composition according to the invention, the nutritionally valuable components are in a ratio suitable for animals, the product is easily rationed, it is tasty and preserves well. The feed supplement composition is a natural product containing vitamins and health-promoting components, which has a high nutrient density. Using carriers, the nutrient density can be decreased, if desired, taking into consideration the needs of the animal, while, at the same time, the different technological characteristics these bring about are of service in the feeding event of the animal.

According to one embodiment, a composition according to the invention is a natural product, to which there is no need to add artificial additives.

The composition has not been observed to have any adverse effects. It is very well tolerated, tasty and easy to use. Regardless of its tastiness, the composition is low-energy; typically, it contains approx. 7% by weight of the daily energy requirements of a 15 kg dog, this is the opposite of many other pet treats on the market, which are high-energy.

The potency/effect of the composition is quickly noticeable, its favourable effects can be observed after less than even a week of use.

The composition contains 20-25% of the daily protein requirements of a 15 kg dog, 20-50% of essential amino acid requirements and approximately 40% of fat requirements, of which fats ⅔ are unsaturated.

The composition naturally contains all the amino acids and fatty acids essential for dogs and cats with the exception of taurine, which is essential for cats. Taurine can, if necessary, be added to the composition or to the daily nourishment of the cat.

The egg fraction contained in an animal feed supplement composition according to the invention has excellent digestibility, typically approximately 92-100 wt % of the egg fraction is utilized and the absorbability of the nutrient contents of the egg fraction is good. Egg fraction contains an abundance of unsaturated fatty acids and it is a good source of DHA. Egg fraction further contains vitamins A, D, B9 and B5 and phosphatidylcholine.

The shell fraction contained in an animal feed supplement composition according to the invention contains minerals, especially calcium, which is important for the health of bones. Calcium absorption from egg shell powder is considerably better than calcium absorption from the limestone obtained from calcium carbonate and, thus, it has an improved effect on the mineral density of the bones. Shell fraction contains organic matrix having valuable transport proteins and glycosaminoglycans (GAG) of the shell as well as calcium and strontium bound in organic compounds. Calcium and strontium are absorbed very well from the shell fraction and increase bone density, strontium promotes the formation of more densely packed bone, thus these have a significant effect in the prevention and treatment of osteoporosis. Previously considered a waste product, shell fraction can thus be utilized as a valuable source of nutrients.

The shell membrane fraction contained in an animal feed supplement composition according to the invention contains collagen, chondroitin sulfate, glucosamine and hyaluronic acid in ratios, which promote the health of cartilage and joints. The shell membrane components increase mobility, decrease pain, they have an anti-inflammatory effect due to the down-regulation of cytokines, such as TNF-α, as well as an effect promoting skin condition.

In a composition according to the invention, all the valuable parts of egg are utilized including the egg fraction, the shell and the shell membrane. In the composition, the components are in an ideal ratio in terms of the daily needs of the animal, due to their natural form they have considerably better digestibility and absorbability, thus their biological availability is high. Due to this, for animals do not need to be rationed the ordinarily-used high rations of dietary supplements etc., which often cause undesired side effects.

The composition is an excellent source of vitamins, it contains i.a. vitamin A, vitamin D, vitamin E, which also function as antioxidants affecting preservability of the product, vitamins B2, B5, B12 and folate, an abundance of choline, which is important i.a. for the skin, liver function and neurological functions of the animal, lutein and selenium, which is well absorbed.

The Ca/P ratio of an animal supplemental feed composition according to the invention is optimal for the health of the animal and the ratio can be adjusted within given limits as suitable for each animal species.

The tasty and well-preservable animal feed supplement composition according to the invention can be given to animals as an addition to their daily nourishment to nutritionally supplement an adequate supply of important components to the animal in the correct ratio and in as efficient form as possible in terms of utilization.

An animal feed supplement composition according to the invention contains natural components originating from egg that have excellent digestibility and absorbability, so the composition is, in most cases, also well-suited for animals suffering from allergies or hypersensitivities.

An animal feed supplement composition according to the invention can be given to pregnant and nursing animals as well as to young or growing animals to support the optimal development of the animal, because the composition contains i.a. choline required by brains and nervous system, easily-absorbed calcium and phosphorus required for development of bones, an abundance of proteins. The fat of the feed supplement is soft and it contains omega-6 and omega-3 fatty acids, such as DHA and a small amount of EPA. DHA is especially significant in the development of brains and eyesight.

An animal feed supplement composition according to the invention is also an excellent addition to the nutritional requirements of animals that do hard physical labour, are physically active and aging animals, such as dogs and cats, because the composition contains shell membrane fraction advantageous for the health of joints and cartilage, an abundance of proteins, which are important for maintaining muscle mass, and lutein, which is beneficial to the welfare of the eyes. The fat of egg fraction includes natural antioxidants, lutein and vitamins A and D, which together slow the oxidation of fat. Preventing fat oxidation also naturally slows the oxidation reactions of other nutrient components (proteins, sulphur compounds) caused by the oxidation products of fat.

The composition has been observed to favourably affect both the physical health and the mental agility of an animal.

The following example illustrates embodiments according to the invention described above, and it is not intended to limit the invention in any way.

### EXAMPLES

### Example 1

Preparation of the Animal Feed Supplement Composition

The composition powder was prepared by mixing dried egg fraction containing chicken egg albumen and yolk with shell fraction and shell membrane fraction of chicken egg in a ratio of 95:3:2 according to weight. The mixing was performed with a mixing device suitable for mixing dry, powder-like substances. The order and time of the mixing was selected such that the composition became homogeneous. The preparation lot was 5 kg.

The composition powder was granulated immediately after mixing with a device for feed granulation. The granules were packed into canisters made of PET plastic, which were closed and stored in the cold (+5° C.).

### Example 2

Preparation and Preservability of the Animal Feed Supplement Composition

The composition powder was prepared by mixing dried egg fraction containing chicken egg albumen and yolk (whole egg powder) with shell fraction (shell powder) and shell membrane fraction (membrane powder) of chicken egg in a ratio of 95:3:2 according to weight. The mixing was performed with a mixing device suitable for mixing dry, powder-like substances. The order and time of the mixing was selected such that the composition became homogeneous. Two preparation lots were prepared: one 8 kg (composition A) and the other 7.5 kg (composition B).

The composition powder was granulated immediately after mixing with a device for feed granulation, from which were obtained extruded oblong granules. The granules were packed into canisters made of PET and HDPE plastic, which were closed and stored in the cold (+5° C.).

The dry matter content of the granules was 95.6 wt % (A) and 95.3 wt % (B) and a salmonella test was negative.

The preservability of the granules were monitored at 25° C./RH 45% in a 6 month test. The preservability and microbiological quality were good and no significant changes were observed.

### Example 3

Research of the Animal Feed Supplement Composition in Dogs

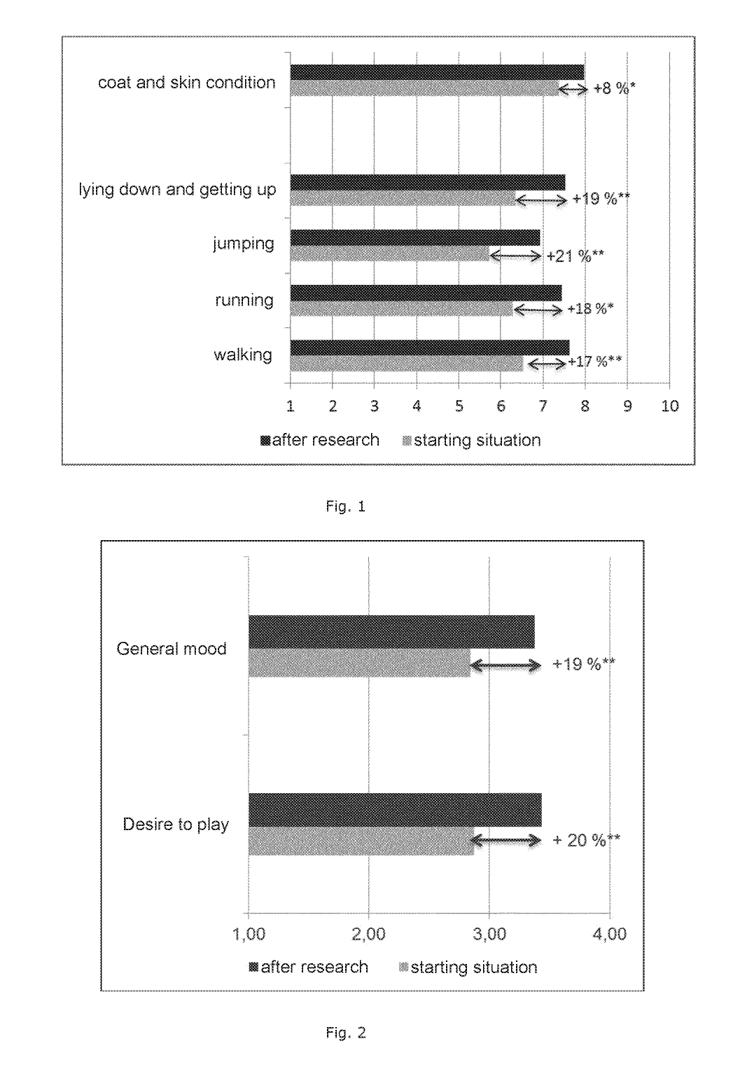

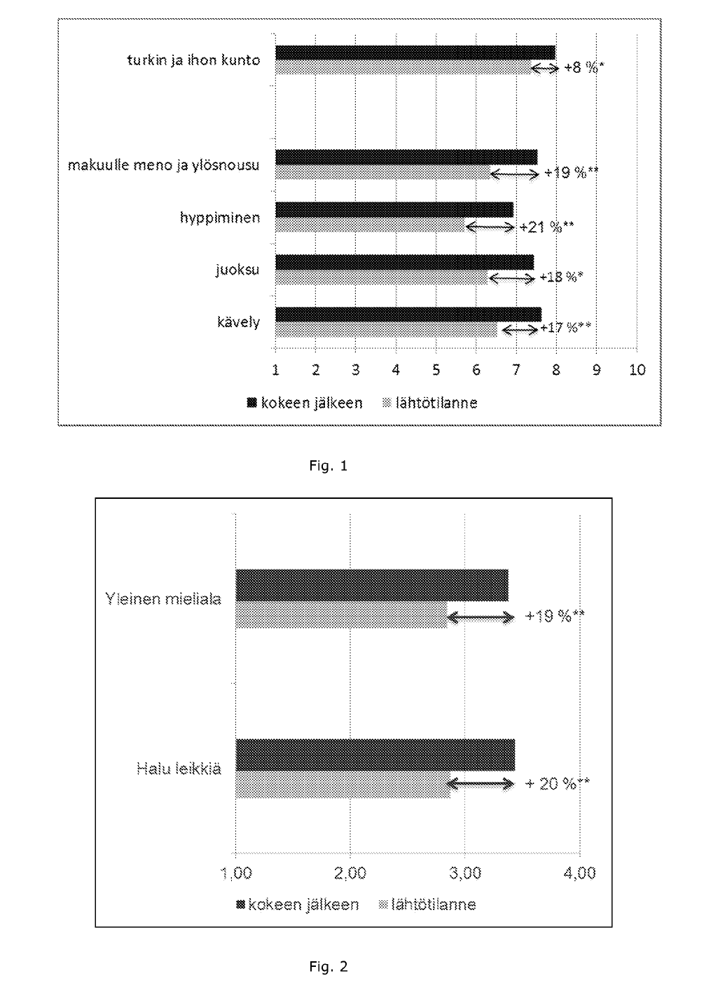

The compositions used in the research are the compositions A and B prepared in Example 2. The effects of the composition were researched in 17 dogs for a 21-day period of time. The age of the dogs varied in the range of 1-14 yrs, included in the research were 6 bitches and 11 males. The composition was rationed for dogs less than 10 kg 2 tsp/day, dogs 10-45 kg 2 tbsp/day and dogs over 45 kg 3 tbsp/day. The mobility, coat/skin condition of the dogs, the desire to play and the general mood of the dog were monitored during the period (3 weeks) the composition was rationed. The results are shown graphically in FIGS. 1 and 2.

FIG. 1 shows the effect of the composition on the mobility and coat/skin condition of the dog. FIG. 2 shows the effect of the composition on the desire to play and general mood of the dog.

On the basis of the results illustrated in the figures, it can be seen that the composition according to the invention positively affects the mobility, desire to play, general mood as well as the coat and skin condition of the dogs.

The composition was found to be very tasty and no digestive problems were expressed in the animals.

1-17. (canceled)

18. An animal feed supplement composition, wherein the feed supplement composition comprises 80-98 wt % of dried egg fraction composed of yolk and albumen separated from raw poultry eggs, 0.1-15 wt % of shell membrane fraction and 1-10 wt % of shell fraction.

19. An animal feed supplement composition according to claim 18, wherein the poultry is selected from bird species grown or ranched for the production of meat, eggs or other products, messenger, show and ornamental bird species as well as game bird species.

20. An animal feed supplement composition according to claim 18, wherein the animals are selected from companion animals, fur animals and production animals, preferably the companion animals are selected from dogs, cats, ferrets, rodents, pigs, turtles, reptiles and birds, preferably fur animals are selected from foxes, minks, chinchillas, nutrias, raccoons, sables, ermines, muskrats and polecats, and preferably production animals are selected from bovines, hoofed animals, llamas, alpacas, sheep, goats and pigs.

21. An animal feed supplement composition according to claim 18, wherein the feed supplement composition comprises 91-98 wt % of dried egg fraction composed of yolk and albumen separated from raw poultry eggs, 0.5-5.5 wt % of shell membrane fraction and 1.5-3.5 wt % of shell fraction.

22. An animal feed supplement composition according to claim 18, wherein the feed supplement composition comprises 92.5-97 wt % of dried egg fraction composed of yolk and albumen separated from raw poultry eggs, 1-4 wt % of shell membrane fraction and 2-3.5 wt % of shell fraction.

23. An animal feed supplement composition according to claim 18, wherein the shell fraction is selected from the shell separated from the poultry egg, organic calcium salt, inorganic calcium carbonate and their combinations.

24. An animal feed supplement composition according to claim 18, wherein the ratio of calcium to phosphorus of the composition is in the range of 1:1-2:1.

25. A product containing an animal feed supplement composition according to claim 18 and one or more additives.

26. A product according to claim 25, wherein the additive is selected from antioxidants, flavouring agents, chondroprotectives, vitamins and vitamin sources, minerals and trace elements, processing aids, the oils and fractions obtained from fish and other seafood, algae, plankton, vegetable oils and their combinations.

27. A product according to claim 25, wherein the additive is vitamin C, vitamin E or their combinations.

28. A product according to claim 25, wherein the product is in the form of powder, granules, pellets, tablets, capsules, gel, paste, emulsion, liquid, chew bars, chew bones, chew rolls or treats.

29. A method for preparing the animal feed supplement composition according to claim 18, wherein, in the method, 80-98 wt % of dried egg fraction composed of yolk and albumen separated from raw poultry eggs, 0.1-15 wt % of shell membrane fraction and 1-10 wt % of shell fraction, are combined into a homogenous mixture.

30. A method according to claim 29, wherein, in the method, 91-98 wt % of dried egg fraction composed of yolk and albumen separated from raw poultry eggs, 0.5-5.5 wt % of shell membrane fraction and 1.5-3.5 wt % of shell fraction, are combined.

31. A method according to claim 29, wherein, in the method, 92.5-97 wt % of dried egg fraction composed of yolk and albumen separated from raw poultry eggs, 1-4 wt % of shell membrane fraction and 2-3.5 wt % of shell fraction, are combined.

32. A method according to claim 29, wherein additives are mixed into the prepared composition or they are mixed into the composition in connection with preparation.

33. A method according to claim 29, wherein the additive is selected from antioxidants, flavouring agents, chondroprotectives, vitamins and vitamin sources, minerals and trace elements, processing aids, the oils and fractions obtained from fish and other seafood, algae, plankton, vegetable oils and their combinations.

34. A method according to claim 29, wherein from the mixture are prepared products comprising the feed supplement composition, which are in the form of powder, granules, pellets, tablets, capsules, gel, paste, emulsion or liquid or the mixture is mixed into a carrier and from it are made chew bars, chew bones, chew rolls, treats, or the mixture is combined into animal food or drink.

35. An animal feed supplement composition according to claim 19, wherein the animals are selected from companion animals, fur animals and production animals, preferably the companion animals are selected from dogs, cats, ferrets, rodents, pigs, turtles, reptiles and birds, preferably fur animals are selected from foxes, minks, chinchillas, nutrias, raccoons, sables, ermines, muskrats and polecats, and preferably production animals are selected from bovines, hoofed animals, llamas, alpacas, sheep, goats and pigs.

36. An animal feed supplement composition according to claim 19, wherein the feed supplement composition comprises 91-98 wt % of dried egg fraction composed of yolk and albumen separated from raw poultry eggs, 0.5-5.5 wt % of shell membrane fraction and 1.5-3.5 wt % of shell fraction.

37. An animal feed supplement composition according to claim 20, wherein the feed supplement composition comprises 91-98 wt % of dried egg fraction composed of yolk and albumen separated from raw poultry eggs, 0.5-5.5 wt % of shell membrane fraction and 1.5-3.5 wt % of shell fraction.
